(19) United States
(12) Patent Application Publication
(10) Pub. No.: US 2012/0046710 A1
(43) Pub. Date: Feb. 23, 2012

(54) METHODS, SYSTEMS, AND DEVICES FOR DEEP BRAIN STIMULATION USING HELICAL MOVEMENT OF THE CENTROID OF STIMULATION

(75) Inventors: Andrew DiGiore, San Francisco, CA (US); David Karl Lee Peterson, Valencia, CA (US)

(73) Assignee: Boston Scientific Neuromodulation Corporation, Valencia, CA (US)

(21) Appl. No.: 13/198,035

(22) Filed: Aug. 4, 2011

Related U.S. Application Data
(60) Provisional application No. 61/374,883, filed on Aug. 18, 2010.

(51) Int. Cl. A61N 1/36

(52) U.S. Cl. 607/45

(57) ABSTRACT

A method of treating a target region in the brain includes a) contacting tissue to be stimulated with a lead of a stimulation device, the stimulation device comprising a pulse generator coupled to the lead, the lead having a plurality of segmented electrodes disposed at a distal end of the lead, the stimulation device being configured and arranged to stimulate a target region using a positionable centroid of stimulation; b) providing stimulation current to at least one of the segmented electrodes of the lead to generate a centroid of stimulation at a location and stimulate tissue around the location of the centroid of stimulation; c) repositioning the centroid of stimulation to a next location along a helical path by altering the provision of stimulation current to the plurality of electrodes and stimulating tissue around the location of the repositioned centroid of stimulation; and d) repeating c) for each location along the helical path. The method may optionally include collecting data associated with each of the locations of the centroid of stimulation; and displaying at least a portion of the collected data.

### CROSS-REFERENCE TO RELATED APPLICATIONS

This application claims the benefit under 35 U.S.C. §119(e) of U.S. Provisional Patent Application Ser. No. 61/374,883 filed on Aug. 18, 2010, which is incorporated herein by reference.

### FIELD

The invention is directed to devices and methods for brain stimulation including deep brain stimulation. In addition, the invention is directed to methods, systems and devices that utilize helical movement of the centroid of stimulation.

### BACKGROUND

Deep brain stimulation can be useful for treating a variety of conditions including, for example, Parkinson's disease, dystonia, essential tremor, chronic pain, Huntington's Disease, levodopa-induced dyskinesias and rigidity, bradykinesia, epilepsy and seizures, eating disorders, and mood disorders. Typically, a lead with a stimulating electrode at or near a tip of the lead provides the stimulation to target neurons in the brain. Magnetic resonance imaging (MRI) or computerized tomography (CT) scans can provide a starting point for determining where the stimulating electrode should be positioned to provide the desired stimulus to the target neurons.

Upon insertion, current is introduced along the length of the lead to stimulate target neurons in the brain. This stimulation is provided by electrodes, typically in the form of rings, disposed on the lead. The current projects from each electrode similarly and in all directions at any given length along the axis of the lead. Because of the shape of the electrodes, radial selectivity of the current is minimal. This results in the unwanted stimulation of neighboring neural tissue, undesired side effects and an increased duration of time for the proper therapeutic effect to be obtained.

### BRIEF SUMMARY

One embodiment is a method of treating a target region in the brain that includes a) contacting tissue to be stimulated with a lead of a stimulation device, the stimulation device comprising a pulse generator coupled to the lead, the lead having a plurality of segmented electrodes disposed at a distal end of the lead, the stimulation device being configured and arranged to stimulate a target region using a positionable centroid of stimulation; b) providing stimulation current to at least one of the segmented electrodes of the lead to generate a centroid of stimulation at a location and stimulate tissue around the location of the centroid of stimulation; c) repositioning the centroid of stimulation to a next location along a helical path by altering the provision of stimulation current to the plurality of electrodes and stimulating tissue around the location of the repositioned centroid of stimulation; and d) repeating c) for each location along the helical path.

The method may optionally include collecting data associated with each of the locations of the centroid of stimulation; and displaying at least a portion of the collected data.

Another embodiment is a computer-readable medium having processor-executable instructions for stimulating tissue. The processor-executable instructions when installed onto a stimulation device enable the stimulation device to perform actions. The stimulation device includes a pulse generator coupleable to a lead having a plurality of segmented electrodes disposed at a distal end of the lead, the stimulation device being configured and arranged to stimulate a target region using a positionable centroid of stimulation. The actions include a) providing stimulation current to at least one of the segmented electrodes of the lead to generate a centroid of stimulation at a location and stimulate tissue around the location of the centroid of stimulation; b) repositioning the centroid of stimulation to a next location along a helical path by altering the provision of stimulation current to the plurality of electrodes and stimulating tissue around the location of the repositioned centroid of stimulation; and c) repeating b) for each location along the helical path.

Yet another embodiment is a stimulation device that includes a pulse generator coupleable to a lead having a plurality of segmented electrodes disposed at a distal end of the lead, the stimulation device being configured and arranged to stimulate a target region using a positionable centroid of stimulation. The stimulation device also includes a processor for executing processor-readable instructions that enable actions. The actions include a) providing stimulation current to at least one of the segmented electrodes of the lead to generate a centroid of stimulation at a location and stimulate tissue around the location of the centroid of stimulation; b) repositioning the centroid of stimulation to a next location along a helical path by altering the provision of stimulation current to the plurality of electrodes and stimulating tissue around the location of the repositioned centroid of stimulation; and c) repeating b) for each location along the helical path.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic perspective view of one embodiment of a portion of a lead having a plurality of segmented electrodes and a ring electrode, according to the invention;

FIG. 1B is a schematic perspective view of another embodiment of a lead having a plurality of segmented electrodes arranged in staggered orientation and a ring electrode, according to the invention;

FIG. 2A is a schematic diagram of radial current steering along various electrode levels along the length of a lead, according to the invention;

FIG. 2B is a schematic diagram of one embodiment of stimulation volume using monopolar and multipolar stimulation techniques, according to the invention;

FIG. 3 is a schematic representation of a device for deep brain stimulation, according to the invention;

FIG. 4A is a schematic perspective view of conventional current steering;

FIG. 4B is a schematic perspective view of one embodiment of current steering, according to the invention;

FIG. 4C is a schematic perspective view of a second embodiment of current steering, according to the invention;

FIG. 4D is a schematic perspective view of a third embodiment of current steering, according to the invention;

FIG. 5A is a schematic cross-sectional view of a conventional stimulation profile;

FIG. 5B is a schematic cross-sectional view of one embodiment of a stimulation profile, according to the invention;

FIG. 6 is a flow-chart of one embodiment of a method, according to the invention;

FIG. 7 is a flow-chart of a second embodiment of a method, according to the invention;

FIG. 8 is a schematic side view of one embodiment of a device for brain stimulation, according to the invention.

### DETAILED DESCRIPTION

The present invention is directed to the area of devices and methods for brain stimulation including deep brain stimulation. In addition, the invention is directed to devices and methods for brain stimulation using a lead having a plurality of segmented electrodes arranged in a ring array, and methods of helical movement of the centroid of stimulation using such devices.

A lead for deep brain stimulation may include stimulation electrodes, recording electrodes, or a combination of both. A practitioner may determine the position of the target neurons using the recording electrode(s) and then position the stimulation electrode(s) accordingly without removal of a recording lead and insertion of a stimulation lead. In some embodiments, the same electrodes can be used for both recording and stimulation. In some embodiments, separate leads can be used; one with recording electrodes which identify target neurons, and a second lead with stimulation electrodes that replaces the first after target neuron identification. A lead may include recording electrodes spaced around the circumference of the lead to more precisely determine the position of the target neurons. In at least some embodiments, the lead is rotatable so that the stimulation electrodes can be aligned with the target neurons after the neurons have been located using the recording electrodes.

Deep brain stimulation devices and leads are described in the art. See, for instance, U.S. Pat. No. 7,809,446 (“Devices and Methods For Brain Stimulation”), and U.S. Patent Application Publication No. 2010/0076535 (“Leads With Non-Circular-Shaped Distal Ends For Brain Stimulation Systems and Methods of Making and Using”). Each of these references is incorporated herein by reference in its respective entirety.

In the field of deep brain stimulation, radially segmented electrode arrays (RSEA) have been developed to provide superior radial selectivity of current. Radially segmented electrode arrays are useful for deep brain stimulation because the target structures in the deep brain are often not symmetric about the axis of the distal electrode array. In some cases, a target may be located on one side of a plane running through the axis of the lead. In other cases, a target may be located at a plane that is offset at some angle from the axis of the lead. Thus, radially segmented electrode arrays may be useful for selectively simulating tissue.

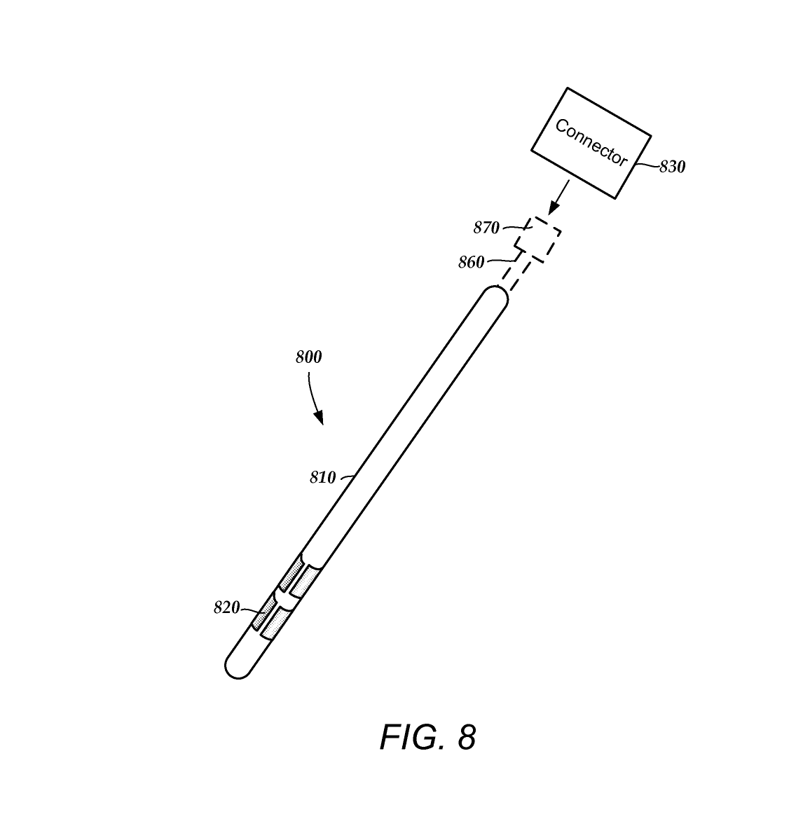

FIG. 8 illustrates one embodiment of a device 800 for brain stimulation. The device includes a lead 810, a plurality of segmented electrodes 820 disposed about the lead body 810, a connector 830 for connection of the electrodes to a control unit, and a stylet 860 for assisting in insertion and positioning of the lead in the patient's brain. The stylet 860 can be made of a rigid material. Examples of suitable materials include tungsten, stainless steel, or plastic. The stylet 860 may have a handle 870 to assist insertion into the lead, as well as rotation of the stylet and lead. The connector 830 fits over the proximal end of the lead 810, preferably after removal of the stylet 860.

In one example of operation, access to the desired position in the brain can be accomplished by drilling a hole in the patient's skull or cranium with a cranial drill (commonly referred to as a burr), and coagulating and incising the dura mater, or brain covering. The lead 810 can be inserted into the cranium and brain tissue with the assistance of the stylet 860. The lead can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): rotate the lead, insert the lead, or retract the lead. In some embodiments, measurement devices coupled to the muscles or other tissues stimulated by the target neurons or a unit responsive to the patient or clinician can be coupled to the control unit or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in tremor frequency or amplitude in response to stimulation of neurons. Alternatively, the patient or clinician may observe the muscle and provide feedback.

It will be understood that the lead 810 for deep brain stimulation can include stimulation electrodes, recording electrodes, or both. In at least some embodiments, the lead is rotatable so that the stimulation electrodes can be aligned with the target neurons after the neurons have been located using the recording electrodes. Alternatively or additionally, an array of electrodes is provided so that electrodes with the desired alignment (e.g., location on the lead) can be used.

Stimulation electrodes may be disposed on the circumference of the lead to stimulate the target neurons. Stimulation electrodes may be ring-shaped so that current projects from each electrode equally in every direction at any given length along the axis of the lead. To achieve current steering, segmented electrodes can be utilized additionally or alternatively. Though the following description discusses stimulation electrodes, it will be understood that all configurations of the stimulation electrodes discussed may be utilized in arranging recording electrodes as well, including, for example, ring electrodes, segmented electrodes, or combinations thereof.

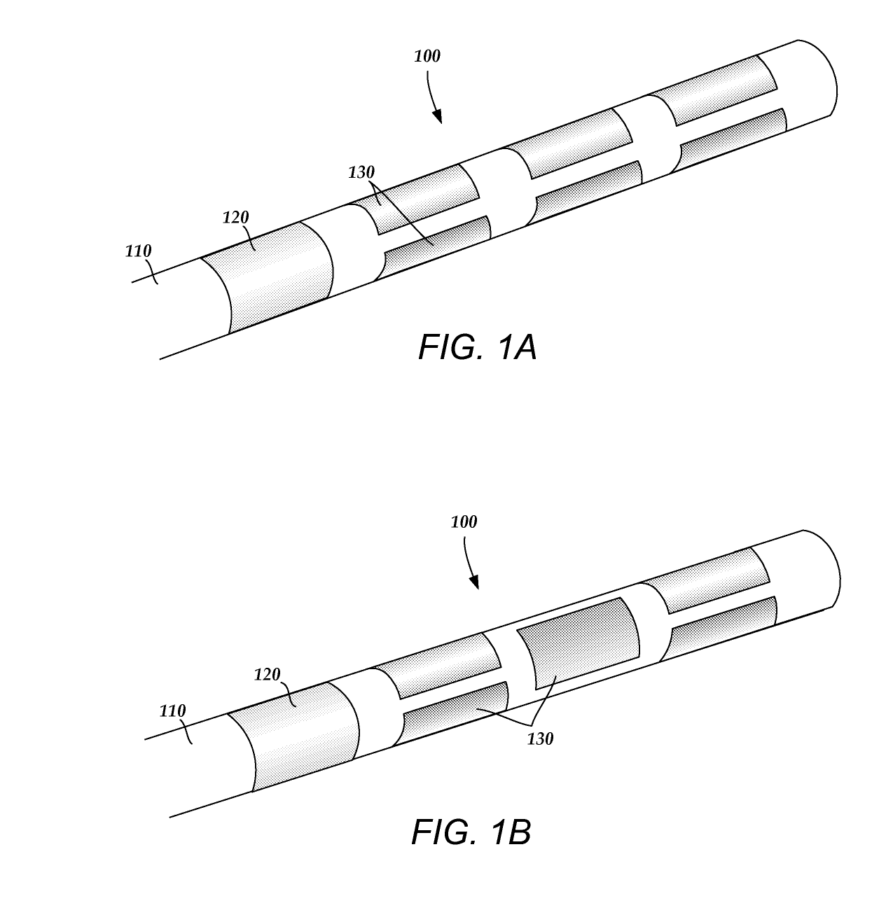

FIG. 1A illustrates one embodiment of a lead 100 for brain stimulation. The device includes a lead body 110, one or more ring electrodes 120, and a plurality of segmented electrodes 130. The lead body 110 can be formed of a biocompatible, non-conducting material such as, for example, a polymeric material. Suitable polymeric materials include, but are not limited to, silicone, polyethylene, polyurethanes, polyureas, or polyurethane-ureas. In at least some instances, the lead may be in contact with body tissue for extended periods of time. In at least some embodiments, the lead has a cross-sectional diameter of no more than 1.5 mm and may be in the range of 0.75 to 1.5 mm. In at least some embodiments, the lead has a length of at least 10 cm and the length of the lead may be in the range of 25 to 70 cm.

Stimulation electrodes may be disposed on the lead body 110. These stimulation electrodes may be made using a metal, alloy, conductive oxide, or any other suitable conductive material. Examples of suitable materials include, but are not limited to, platinum, iridium, platinum iridium alloy, stainless steel, titanium, palladium, or tungsten. Preferably, the stimulation electrodes are made of a material that is biocompatible and does not substantially corrode under expected operating conditions in the operating environment for the expected duration of use.

In at least some embodiments, any of the electrodes can be used as an anode or cathode and carry anodic or cathodic current. In some instances, an electrode might be an anode for a period of time and a cathode for a period of time. In other embodiments, the identity of a particular electrode or electrodes as an anode or cathode might be fixed.

The lead contains a plurality of segmented electrodes 130. Any number of segmented electrodes 130 may be disposed on the lead body 110. In some embodiments, the segmented electrodes 130 are grouped in sets of segmented electrodes, each set disposed around the circumference of the lead at or near a particular longitudinal position. The lead may have any number of sets of segmented electrodes. In at least some embodiments, the lead has one, two, three, four, five, six, seven, or eight sets of segmented electrodes. In at least some embodiments, each set of segmented electrodes contains the same number of segmented electrodes 130. Alternatively, one or more of the sets of segmented electrodes can have a different number of electrodes 130 from the other sets of electrodes. In some embodiments, each set of segmented electrodes contains three segmented electrodes 130. In at least some other embodiments, each set of segmented electrodes contains one (e.g., an electrode that only forms a portion of the circumference of the lead), two, four, five, six, seven or eight segmented electrodes. In one embodiment, there are four sets of segmented electrodes with two electrode, three electrodes, three electrodes, and two electrodes, respectively (a 2-3-3-2 arrangement). In another embodiment, there are four sets of segmented electrodes, with three, four, four, and three electrodes respectively, flanked by ring electrodes on either end (a 1-3-4-4-3-1 arrangement). In yet another embodiment, there are two sets of three segmented electrodes each flanked by ring electrodes on either end (a 1-3-3-1 arrangement).

The segmented electrodes 130 may vary in size and shape. For example, in FIG. 1B, the segmented electrodes 130 are shown as portions of a ring or curved rectangular portions. In some other embodiments, the segmented electrodes 130 are curved square portions. The shape of the segmented electrodes 130 may also be substantially triangular, diamond-shaped, oval, circular or spherical. In some embodiments, the segmented electrodes 130 are all of the same size, shape, diameter, width or area or any combination thereof. In some embodiments, the segmented electrodes of each set (or even all segmented electrodes) may be identical in size and shape.

In at least some embodiments, each set of segmented electrodes 130 may be disposed around the circumference of the lead body 110 to form a substantially or approximately cylindrical shape around the lead body 110. The spacing of the segmented electrodes 130 around the circumference of the lead body 110 may vary. In at least some embodiments, equal spaces, gaps or cutouts are disposed between each segmented electrodes 130 around the circumference of the lead body 110. In other embodiments, the spaces, gaps or cutouts between segmented electrodes may differ in size or shape. In other embodiments, the spaces, gaps, or cutouts between segmented electrodes may be uniform for a particular set of segmented electrodes or for all sets of segmented electrodes. The segmented electrodes 130 may be positioned in irregular or regular intervals around the lead body 110.

As indicated in examples above, stimulation electrodes in the form of ring electrodes 120 may be disposed on any part of the lead body 110, usually near a distal end of the lead. FIG. 1A illustrates a portion of a lead having one ring electrode. Any number of ring electrodes may be disposed along the length of the lead body 110. For example, the lead body may have one ring electrode, two ring electrodes, three ring electrodes or four ring electrodes. In some embodiments, the lead will have five, six, seven or eight ring electrodes. Other embodiments do not include ring electrodes.

In some embodiments, the ring electrodes 120 are substantially cylindrical and wrap around the entire circumference of the lead body 110. In some embodiments, the outer diameter of the ring electrodes 120 is substantially equal to the outer diameter of the lead body 110. Furthermore, the width of ring electrodes 120 may vary according to the desired treatment and the location of the target neurons. In some embodiments the width of the ring electrode 120 is less than or equal to the diameter of the ring electrode 120. In other embodiments, the width of the ring electrode 120 is greater than the diameter of the ring electrode 120.

Conductors (not shown) that attach to or from the ring electrodes 120 and segmented electrodes 130 also pass through the lead body 110. These conductors may pass through the material of the lead or through a lumen defined by the lead. The conductors are presented at a connector for coupling of the electrodes to a control unit (not shown). In one embodiment, the stimulation electrodes correspond to wire conductors that extend out of the lead body 110 and are then trimmed or ground down flush with the lead surface. The conductors may be coupled to a control unit to provide stimulation signals, often in the form of pulses, to the stimulation electrodes.

FIG. 1B is a schematic perspective view of another embodiment of a lead having a plurality of segmented electrodes. As seen in FIG. 1B, the plurality of segmented electrodes 130 may be arranged in different orientations relative to each other. In contrast to FIG. 1A, where the three sets of segmented electrodes are aligned along the length of the lead body 110, FIG. 1B displays another embodiment in which the three sets of segmented electrodes 130 are staggered. In at least some embodiments, the sets of segmented electrodes are staggered such that no segmented electrodes are aligned along the length of the lead body 110. In some embodiments, the segmented electrodes may be staggered so that at least one of the segmented electrodes is aligned with another segmented electrode of a different set, and the other segmented electrodes are not aligned. For example, if there are four sets of segmented electrodes, the first and third sets may be aligned and the second and fourth sets are also aligned but staggered with respect to the first and third sets.

Any number of segmented electrodes 130 may be disposed on the lead body 110 in any number of sets. FIGS. 1A and 1B illustrate embodiments including three sets of segmented electrodes. These three sets of segmented electrodes 130 may be disposed in different configurations. For example, three sets of segmented electrodes 130 may be disposed on the distal end of the lead body 110, distal to a ring electrode 120. Alternatively, three sets of segmented electrodes 130 may be disposed proximal to a ring electrode 120. By varying the location of the segmented electrodes 130, different coverage of the target neurons may be selected. For example, a specific configuration may be useful if the physician anticipates that the neural target will be closer to the distal tip of the lead body 110, while another arrangement may be useful if the physician anticipates that the neural target will be closer to the proximal end of the lead body 110. In at least some embodiments, the ring electrodes 120 alternate with sets of segmented electrodes 130.

Any combination of ring electrodes 120 and segmented electrodes 130 may be disposed on the lead. In some embodiments the segmented electrodes are arranged in sets. For example, a lead may include a first ring electrode 120, two sets of segmented electrodes, each set formed of three segmented electrodes 130, and a final ring electrode 120 at the end of the lead. This configuration may simply be referred to as a 1-3-3-1 configuration. It may be useful to refer to the electrodes with this shorthand notation. Other eight electrode configurations include, for example, a 2-2-2-2 configuration, where four sets of segmented electrodes are disposed on the lead, and a 4-4 configuration, where two sets of segmented electrodes, each having four segmented electrodes 130 are disposed on the lead. In some embodiments, the lead will have 16 electrodes. Possible configurations for a 16-electrode lead include, but are not limited to 2-3-3-2 (optionally, one or both of the end sets have the two electrodes electrically connected (i.e., ganged)), 4-4-4-4, 8-8, 3-3-3-3-3-1 (and all rearrangements of this configuration), and 2-2-2-2-2-2-2-2.

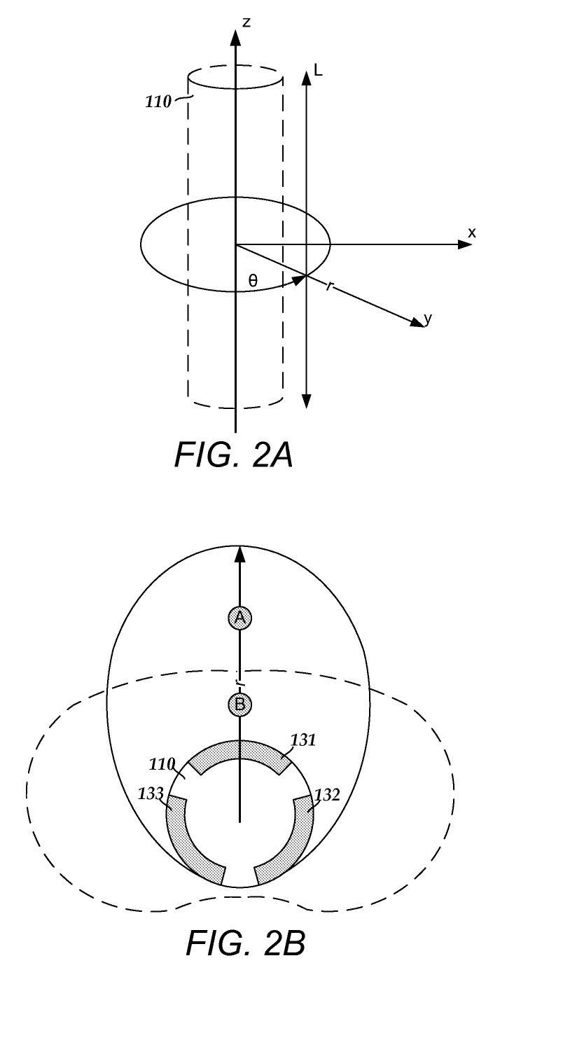

FIG. 2A is a schematic diagram to illustrate radial current steering along various electrode levels along the length of a lead. While conventional lead configurations with ring electrodes are only able to steer current along the length of the lead (the z-axis), the segmented electrode configuration is capable of steering current in the x-axis, y-axis as well as the z-axis. Thus, the centroid of stimulation may be steered in any direction in the three-dimensional space surrounding the lead body 110. In some embodiments, the radial distance, r, and the angle θ around the circumference of the lead body 110 may be dictated by the percentage of anodic current (recognizing that stimulation predominantly occurs near the cathode, although strong anodes may cause stimulation as well) introduced to each electrode as will be described in greater detail below. In at least some embodiments, the configuration of anodes and cathodes along the segmented electrodes 130 allows the centroid of stimulation to be shifted to a variety of different locations along the lead body 110.

As can be appreciated from FIG. 2A, the centroid of stimulation can be shifted at each level along the length of the lead. The use of multiple sets of segmented electrodes 130 at different levels along the length of the lead allows for three-dimensional current steering. In some embodiments, the sets of segmented electrodes 130 are shifted collectively (i.e. the centroid of simulation is similar at each level along the length of the lead). In at least some other embodiments, each set of segmented electrodes 130 is controlled independently. Each set of segmented electrodes may contain two, three, four, five, six, seven, eight or more segmented electrodes. It will be understood that different stimulation profiles may be produced by varying the number of segmented electrodes at each level. For example, when each set of segmented electrodes includes only two segmented electrodes, uniformly distributed gaps (inability to stimulate selectively) may be formed in the stimulation profile. In some embodiments, at least three segmented electrodes 130 are utilized to allow for true 360° selectivity.

In addition to 360° selectivity, a lead having segmented electrodes may provide several advantages. First, the lead may provide for more directed stimulation, as well as less “wasted” or unwanted stimulation (i.e. stimulation of regions other than the target region). By directing stimulation toward the target tissue, side effects may be reduced. Furthermore, because stimulation is directed toward the target site, the battery in an implantable pulse generator may last for a longer period of time between recharging. Moreover, reducing unwanted stimulation may reduce side effects.

Any type of stimulation technique can be used including monopolar stimulation techniques, bipolar stimulation techniques, and multipolar stimulation techniques. FIG. 2B is a schematic diagram of stimulation volume using monopolar and multipolar stimulation techniques. In monopolar stimulation techniques, all local electrodes are of the same polarity (i.e., an electrode of a different polarity is positioned far away and does not affect the stimulation field and centroid; for example an electrode placed on the skin of the patient or the case of an implantable pulse generator used as an electrode). Therefore, the stimulation centroid stays close to the stimulation electrode 131 as represented by B in FIG. 2B. However, in multipolar stimulation, local anode(s) and cathode(s) are used. Therefore, the stimulation field is “driven away” from the electrodes, pushing out the stimulation centroid along the radius r. The centroid of the multipolar stimulation field is represented by A. Note that stimulation amplitude may need to be increased when switching from monopolar to multipolar to keep the same activation volume. As seen in FIG. 2B, the stimulation volume varies between monopolar stimulation, represented by dashed lines, and multipolar stimulation, represented by solid lines. The centroid of the stimulation volume moves out along r when stimulation is changed from monopolar to multipolar. In FIG. 2B, the first segmented electrode 131 is used as the cathode and the second segmented electrodes 132 and 133 are used as anodes in the multipolar configuration, although any other configuration of anode and cathode is possible. Nerve fibers considered were perpendicular to the lead for the purpose of estimating the region of activation. It is recognized that cathodes, and more particularly anodes, may have a stimulating effect for other fiber orientations.

In at least some embodiments, the shift from monopolar stimulation to multipolar stimulation is incremental. For example, a device may start with a cathode (e.g. electrode 131) on the lead and 100% of the anode on the case of the device, or some other nonlocal location. The anode may then be incrementally moved to one or more of the local segmented electrodes 130. Any incremental shift can be used or the shift may even be continuous over a period of time. In some embodiments, the shift is performed in 10% increments. In some other embodiments, the shift is performed in 1%, 2%, 5%, 20%, 25%, or 50% increments. As the anode is incrementally moved from the case to one or more of the segmented electrodes 130, the centroid incrementally moves in the radial direction, r. Table A, below, illustrates an anode shift from a case to one segmented electrode at 10% increments:

TABLE A

| Electrode | Non-Local Anode |
|---|---|
| 0 | 100 |
| 10 | 90 |
| 20 | 80 |
| 30 | 70 |
| 40 | 60 |
| 50 | 50 |
| 60 | 40 |
| 70 | 30 |
| 80 | 20 |
| 90 | 10 |
| 100 | 0 |

Similarly, Table B, below, illustrates an anodic shift from a non-local anode of the device to two segmented electrodes on the lead:

TABLE B

| Electrode 1 | Electrode 2 | Non-Local Anode |
|---|---|---|
| 0 | 0 | 100 |
| 10 | 10 | 80 |
| 20 | 20 | 60 |
| 30 | 30 | 40 |
| 40 | 40 | 20 |
| 50 | 50 | 0 |

In some embodiments, as in Table B, the two segmented electrodes equally split the anode. In other embodiments, the two segmented electrodes unequally split the anode. The two segmented electrodes may also split the anode in any ratio, such as 1.5:1, 2:1 or 3:1.

Another stimulation technique is a method that can be called “chasing the cathode” and can be utilized to project the centroid of the stimulation volume. In this method, the anode chases the cathode around a path of electrodes. It will be recognized that another embodiment can have the cathode chase the anode. After the cathodic current has incrementally shifted to the next segmented electrode, the anodic current begins to incrementally shift to another of the segmented electrodes. Once the anode has completely shifted, the present cathode begins to incrementally shift to the next segmented electrode, and the cycle continues. In at least some embodiments, three or more segmented electrodes are utilized for chasing the cathode. In some cases, the anode shifts may be larger (e.g., 20%) than the cathode shifts (e.g., 10%) or vice versa.

As previously indicated, the foregoing configurations may also be used while utilizing recording electrodes. In some embodiments, measurement devices coupled to the muscles or other tissues stimulated by the target neurons or a unit responsive to the patient or clinician can be coupled to the control unit or microdrive motor system. The measurement device, user, or clinician can indicate a response by the target muscles or other tissues to the stimulation or recording electrodes to further identify the target neurons and facilitate positioning of the stimulation electrodes. For example, if the target neurons are directed to a muscle experiencing tremors, a measurement device can be used to observe the muscle and indicate changes in tremor frequency or amplitude in response to stimulation of neurons. Alternatively, the patient or clinician may observe the muscle and provide feedback.

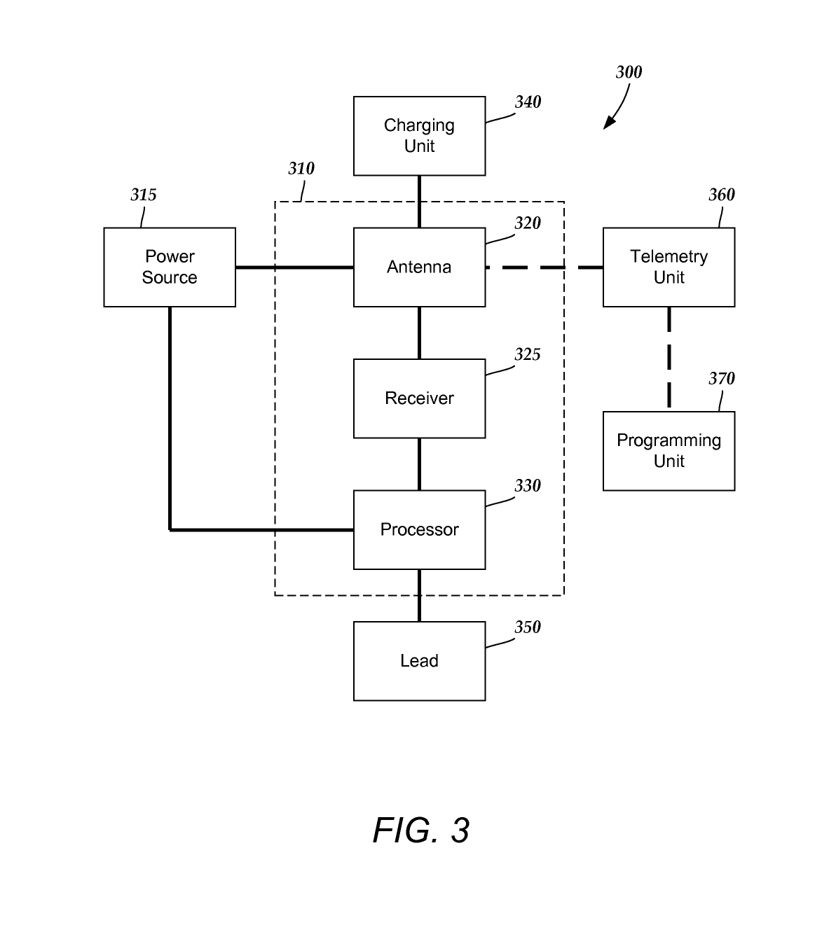

FIG. 3 is a schematic representation of a device for deep brain stimulation 300. It will be understood that the device for deep brain stimulation 300 can include more, fewer, or different components and can have a variety of different configurations including those configurations disclosed in the references cited herein.

The device for brain stimulation 300 may include an implantable pulse generator 310. The implantable pulse generator 310 can include electrical circuitry configured to generate an electrical pulse and a biocompatible casing that houses the electrical circuitry. Some of the components (for example, power source 315, antenna 320, receiver 325, and processor 330) of the device for brain stimulation 300 can be positioned on one or more circuit boards or similar carriers within a sealed housing of an implantable pulse generator of the stimulation device 300, if desired.

In at least some other embodiments, the device for brain stimulation 300 includes an external control unit (not shown) coupled to the lead 350. The external control unit may include electrical circuitry configured to deliver an electrical pulse to the lead 350. With the external control unit coupled to an implanted lead 350, a stimulation profile or parameter of stimulation may be tested without the implantation of a pulse generator 310. Use of the external control unit may also be helpful in finding a target or suitable position for the implantation of the lead 350. It will be understood that the external control unit may include any of the components of the implantable pulse generator 310 as described herein.

Any power source 315 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source 315 through inductive coupling via the optional antenna 320 or a secondary antenna. The external power source 315 can be in a device that is mounted on the skin of the user or in a unit that is provided near the user on a permanent or periodic basis.

In some embodiments, the implantable pulse generator 310 includes a rechargeable battery. In such embodiments, the implantable pulse generator 310 may be coupled to an external charging unit 340 to charge or recharge the rechargeable battery of the implantable pulse generator 310. The battery may be recharged using the optional antenna 320, if desired. In at least some other embodiments, the implantable pulse generator 310 includes a permanent, non-rechargeable battery and an external charging unit 340 is not used.

A lead 350 is coupled to the processor 330 of the implantable pulse generator 310 or the external control unit. The lead 350 may be configured to deliver electrical stimulation signals to one or more structures. The lead 350 may include one more electrodes arranged in any combination as discussed above. In some embodiments, the lead 350 delivers electrical stimulation signals to one or more lobes of the brain such as e.g., the thalamus, subthalamic nucleus, nucleus accumbens, thalamic reticular nucleus, formix, substantia nigra, globus pallidus, and the like. As previously discussed, these stimulation signals may be used to treat various conditions or disorders, including but not limited to, Parkinson's disease, tremor, dyskinesia, obesity, eating disorders, anxiety, depression, Alzheimer's disease, epilepsy, or various other movement disorders. In some embodiments, stimulation may be used to treat multiple disorders concurrently. Moreover, in some embodiments recording electrodes are disposed on the lead to record electrical activity at a specific location.

In some embodiments, the implantable pulse generator 310 includes a processor 330. The processor 330 may be included to control a parameter of stimulation such as, for example, the timing and frequency of stimulation. In addition, in embodiments having multiple leads or independently activated groups of electrodes, the processor 330 can activate any lead or group of electrodes independently or disable it to conserve power. In embodiments having recording electrodes, the processor 330 selects which electrodes are to take a measurement.

Any processor can be used. In some embodiments, the processor 330 is a simple electronic device that produces signals at regular intervals. The processor 330 may also be capable of receiving and interpreting instructions from an external programming unit 370 that, for example, allows modification of signal characteristics. In the illustrated embodiment, the processor 330 is coupled to a receiver 325 which, in turn, is coupled to the optional antenna 320. This allows the processor 330 to receive instructions from an external source to, for example, direct the signal characteristics and the selection of electrodes, if desired.

In some embodiments, the antenna 320 is capable of receiving signals (e.g., RF signals) from an external telemetry unit 360 which is programmed by a programming unit 370. The programming unit 370 can be external to, or part of, the telemetry unit 360. The telemetry unit 360 can be a device that is worn on the skin of the user or can be carried by the user and can have a form similar to a pager, cellular phone, or remote control, if desired. As another alternative, the telemetry unit 360 may not be worn or carried by the user but may only be available at a home station or at a clinician's office. The telemetry unit 360 itself may also be capable of adjusting stimulation parameters of the implantable pulse generator 310. For example, a telemetry unit 360 may be used to adjust the frequency of stimulation. In some embodiments, the telemetry unit 360 is used to adjust the magnitude, duration or location of stimulation as will be described in greater detail below.

The programming unit 370 can be any unit capable of providing information to the telemetry unit 360 for transmission to the device for deep brain stimulation 300. The programming unit 370 can be part of the telemetry unit 360 or can provide signals or information to the telemetry unit 360 via a wireless or wired connection. One example of a suitable programming unit 370 is a computer operated by the user or clinician to send signals to the telemetry unit 360. In some embodiments, the programming unit 370 may be capable of programming and re-programming the implantable pulse generator 310 or the external control unit. Programming of the implantable pulse generator 310 may be done before, during or after implantation of the lead 350.

In some embodiments, the programming unit 370 is a clinician's programmer used to find a set or sets of stimulation parameters for a patient. Stimulation parameters include, but are not limited to, pulse width, amplitude, duration, frequency, burst mode, ramp up time, ramp down time, electrode configuration or any combination thereof.

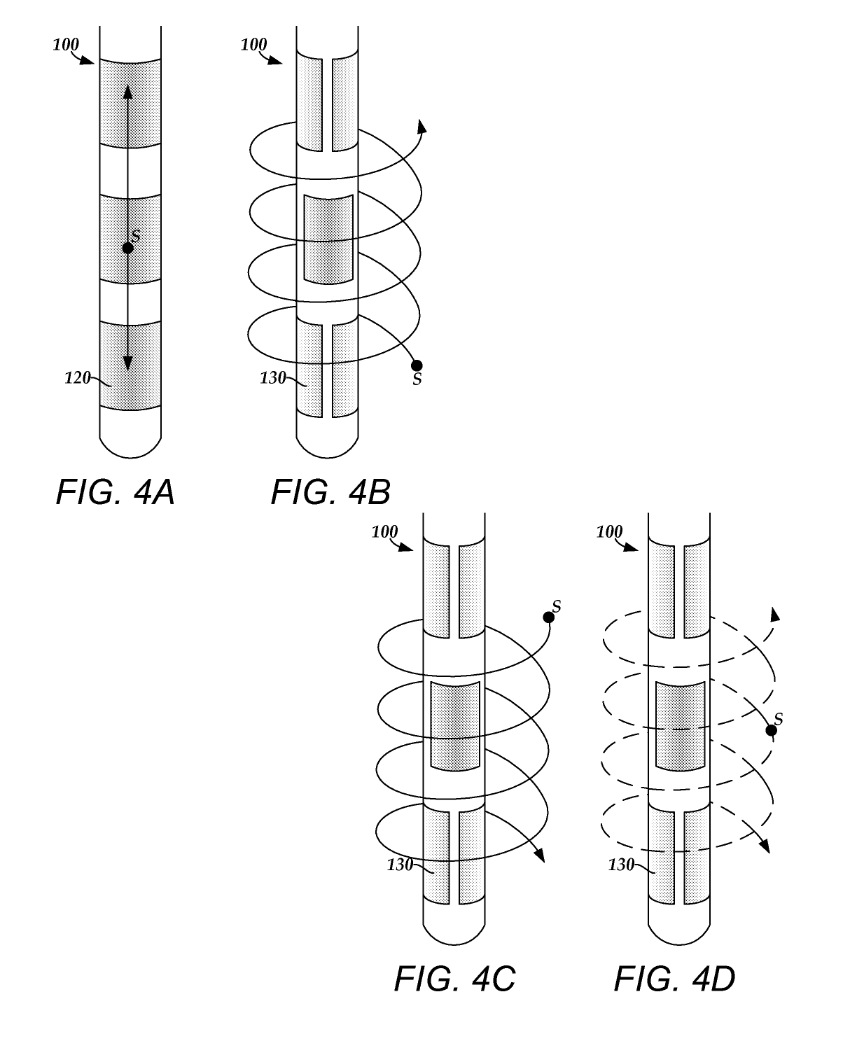

The programming unit 370 may contain software that aids in the programming or operation of the implantable pulse generator 310 or the external control unit. It will be understood that any software referenced herein can also be implemented using hardware or a combination of hardware and software. In some embodiments, the software allows the user to use various methods to program or operate the implantable pulse generator 310 as will be described in greater detail with reference to FIGS. 4A-D.

The signals sent to the processor 330 via the antenna 320 and receiver 325 can be used to modify or otherwise direct the operation of the stimulation device 300. For example, the signals may be used to modify stimulation by adjusting one or more of timing, duration, frequency, magnitude, electrode selection or any combination thereof. The signals may also direct the stimulation device 300 to cease operation, to start operation, to start charging the battery, or to stop charging the battery. In other embodiments, the system does not include an antenna 320 or a receiver 325 and the processor 330 operates as programmed.

Optionally, the stimulation device 300 may include a transmitter (not shown) coupled to the processor 330 and the antenna 320 for transmitting signals back to the telemetry unit 360 or another unit capable of receiving the signals. For example, stimulation device 300 may transmit signals indicating whether the stimulation device 300 is operating properly or not or indicating when the battery needs to be charged or the level of charge remaining in the battery. The processor 330 may also be capable of transmitting information about the pulse characteristics so that a user or clinician can determine or verify the characteristics.

The programming unit 370 may include software that aids in the programming of the implantable pulse generator 310. Software may be used to move the centroid of stimulation in a linear manner, along the axis parallel to the axis of the lead by selection of electrodes. FIG. 4A is a schematic perspective view of this type of current steering. As seen in FIG. 4A, the centroid of stimulation, S, is capable of moving in the longitudinal direction of a lead 100 having ring electrodes 120. As described in FIG. 4A-C, leads having segmented electrodes provide greater possibilities of control.

In some embodiments, the programming unit or processor may be used to move the centroid of stimulation in a spiral-like or helical manner around the axis of the lead 100 by appropriate selection of the segmented electrodes. FIG. 4B is a schematic perspective view of one embodiment of current steering. As seen in FIG. 4B, the lead 100 includes a plurality of segmented electrodes 130. The software of the programming unit 370 may include a method of operating the stimulation device 300 so that the centroid of stimulation, S, moves along a spiral-like or helical path from the distal end of the lead to the proximal end of the lead 100. The centroid of stimulation, S, may travel clockwise or counter-clockwise. It will be understood that the selection of the electrode configuration may also be done manually or may be performed semi-automatically (e.g., where the user inputs one or more parameters, such as starting position or pitch, and the software determines the spiral path).

Movement of the centroid of stimulation, S, may be beneficial in a variety of applications. In some embodiments, the centroid of stimulation, S, is moved during treatment to provide stimulation to one or more target regions in order. In embodiments utilizing recording electrode, the centroid may be shifted to take a measurement at any given site or along any given path. In at least some other embodiments, the centroid of stimulation, S, is moved to find the best set or sets of stimulation parameters for a given target region. By shifting the centroid of stimulation, different sets of stimulation parameters can be compared to find a set that provides effective treatment.

FIG. 4C is a schematic perspective view of a second embodiment of current steering, similar to the current steering of FIG. 4B. The current steering used in FIG. 4C allows the centroid of stimulation, S, to travel in a spiral-like path from the proximal end of the lead 100 to the distal end of the lead 100.

It will be understood that the centroid of stimulation, S, is not limited to movement from one end of the lead 100 to the other end. FIG. 4D provides a schematic perspective view of a third embodiment of current steering. As seen in FIG. 4D, the centroid of stimulation, S, may be programmed to begin stimulation at any point along the lead 100 (e.g. at an intermediate longitudinal position along the length of the lead 100) and may end at any point on the lead. From this intermediate position, the centroid of stimulation, S, may be shifted either to the distal end or the proximal end of the lead 100 or both. Furthermore, the programming unit 370 may be programmed such that the centroid of stimulation begins at one position along the length of the lead 100, shifts toward the proximal end of the lead, and shifts back toward the distal end of the lead 100. The centroid of stimulation may thus be repositioned to any point along the length and about the circumference of the lead as desired. It will be understood that all shifting of the centroid of stimulation may be accomplished in a helical or spiral-like manner, in a linear manner with reference to FIG. 4A, or in a combination of the two methods.

The benefits of the increased flexibility in testing stimulation parameters provided by the embodiments of this invention are numerous. For example, by increasing the programmability of the stimulation device 300, a target region may be more effectively stimulated. Moreover, by increasing the programmability of the stimulation device 300, regions of undesired stimulation may be identified or avoided, reducing side effects as well as prolonging the battery life of the implantable pulse generator 310.

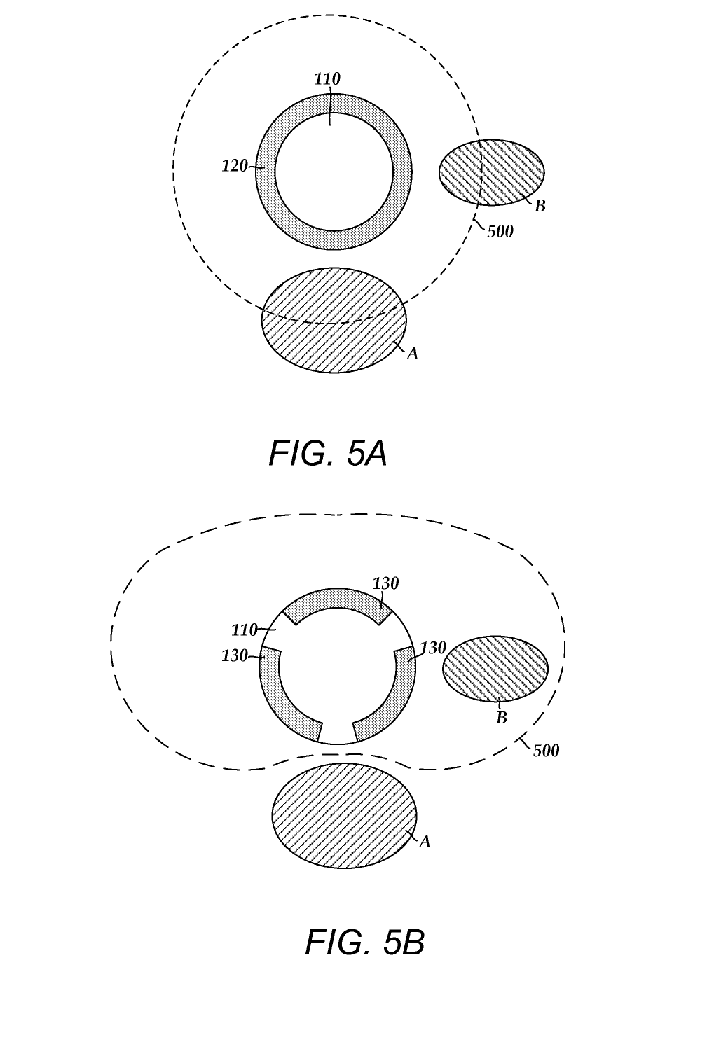

FIG. 5A is a schematic cross-sectional view of a conventional stimulation profile. As seen in FIG. 5A, a ring electrode 120 is disposed about a lead body 110 to produce a region of stimulation 500. As seen in FIG. 5A, the region of stimulation 500 is uniformly arranged about the lead body 110. FIG. 5A also illustrates a region of undesired stimulation, A, and a target region, B. As seen in FIG. 5A, conventional methods unnecessarily stimulate the region of undesired stimulation, A. Stimulating region A may lead to undesired side effects such as dysarthria (slurred speech). Moreover, as seen in FIG. 5A, the region of stimulation 500 does not fully include target region, B, reducing the effectiveness of the treatment.

FIG. 5B is a schematic cross-sectional view of one embodiment of a stimulation profile around a lead body 110 having segmented electrodes 130. As seen in FIG. 5B, the region of stimulation 500 is non-circular and includes a slight upward shift with respect to the lead body 110. In at least some embodiments, the methods described are capable of determining stimulation parameters that can avoid or reduce stimulation of regions of undesired stimulation, A, thus reducing side effects. Moreover, the target region, B, may be more fully enclosed within the region of stimulation 500. In this manner, more effective treatment can be accomplished.

By using the methods described above, more locations and a greater tissue volume may be tested. Moreover, in embodiments having recording electrodes more accurate measurements may be taken. In some embodiments, a region is first stimulated and the results are examined to see whether stimulation should be directed to this region. Using the helical or spiral-like paths discussed above with reference to FIGS. 4B-D, a user has a much greater chance of finding beneficial stimulation parameters. Moreover, the user may more readily avoid areas where stimulation may create or increase side effects. The aforementioned methods allow more tissue volume around the lead 100 to be tested in a structured manner.

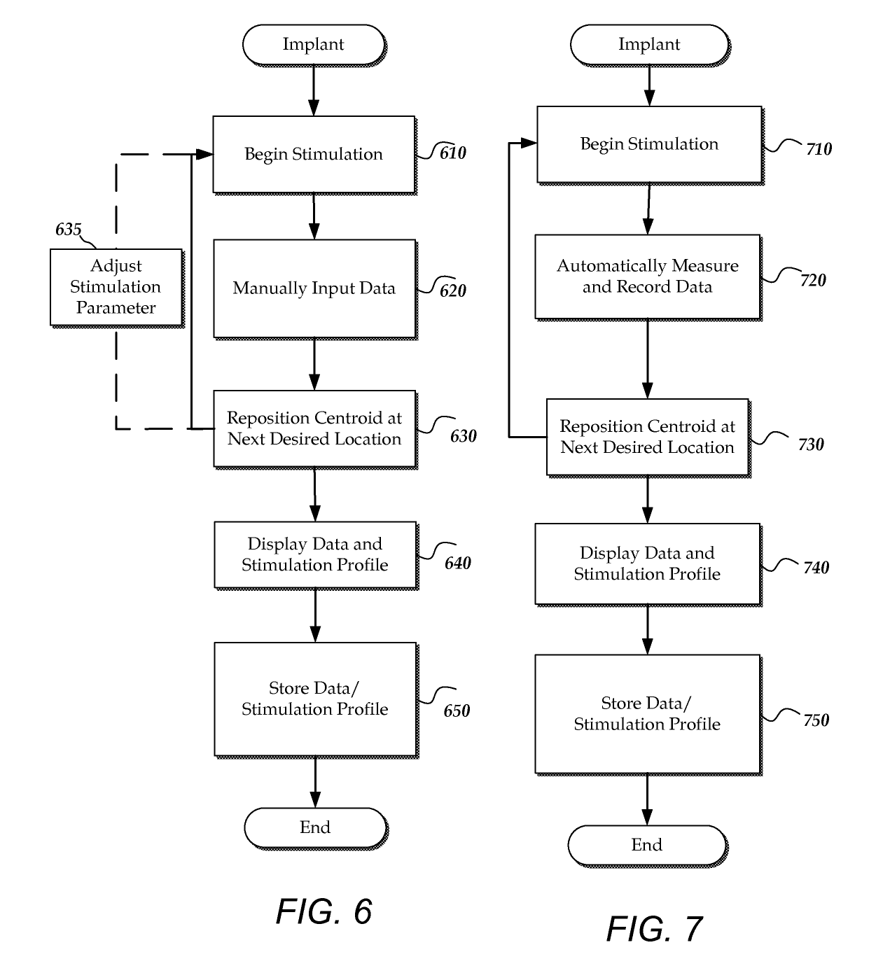

FIG. 6 is a flow-chart of one embodiment of a method of operating a stimulation device 300. As illustrated in FIG. 6, in some embodiments stimulation begins (step 610) after implantation of the lead. Stimulation may begin using any desired set or sets of stimulation parameters at any centroid position as desired. For example, stimulation may begin at a certain position using a user-defined effective electrode configuration, amplitude, frequency, and duration.

In some embodiments, the user manually inputs data (step 620) after stimulation begins. The entered data may include, for example, data relating to side effects, success of therapy, level of paresthesia, level of discomfort, effectiveness of therapy, stimulation level, electrode selection or any combination thereof. In some embodiments, the data is entered at the programming unit 370 as the centroid of stimulation, S, travels along a helical path about a lead 100. For example, stimulation may begin at a certain position and data may be entered corresponding to that position. It will also be understood that some data may be recorded automatically such as stimulation current, electrode selection, stimulation pattern, stimulation duration and the like.

The centroid of stimulation, S, is repositioned to another target position (step 630). In some embodiments, the centroid of stimulation, S, is repositioned after data input at each location. It will be understood that the repositioning of the centroid of stimulation, S, may follow a predefined path selected by the user. For example, in some embodiments, repositioning of the centroid of stimulation, S, involves repositioning the centroid of stimulation, S, at a second point along a spiral-like or helical path. In at least some other embodiments, a linear path is chosen in repositioning the centroid of stimulation, S. A path may also be defined to include both linear and spiral-like features. Repositioning of the centroid of stimulation may also include repositioning the centroid at varying intervals. For example, while traveling along a given helical path, the centroid of stimulation may be shifted to any position along the path so that adjacent positions of stimulation are close together or further apart. Thus, the number of positions along any given paths may be chosen.

Optionally, a stimulation parameter may be adjusted (step 635) after repositioning of the centroid of stimulation. The adjusted stimulation parameter may be any parameter such as amplitude, frequency, pulse width, duration or any combination thereof. In some embodiments, stimulation begins at a first position and data is recorded (step 620), the stimulation parameter is then adjusted and date is recorded and the centroid is then repositioned to a second position. In this manner, two points of data may be collected at each location of stimulation, one using a first stimulation parameter and another using a second stimulation parameter.

Optionally, a portion of the data is arranged and displayed (step 640) in any suitable manner. In some embodiments, the programming unit 370 generates a chart showing the relationship between the stimulation profile and the data entered by the user. In at least some other embodiments, a three-dimensional graph, an image or a plot may be displayed or printed to further evaluate the relationship between stimulation profile and input data. In this manner, a stimulation profile may be selected to have an effective electrode configuration, amplitude, frequency, pulse width, duration, burst mode, ramp up time, ramp down time, or any combination thereof.

Optionally, data may be stored in a memory (step 650) before or after display, or even without display of the data. Any type data may be stored in the memory such as data relating to the stimulation parameters, raw data entered by the user (e.g. side effects and the like), generated displays (e.g. a chart, plot or graph) or any combination thereof. In some embodiments, the data is stored in a memory for future reference or analysis.

It will be understood that the lead may also be used to provide stimulation as therapy with the repositioning of the centroid of stimulation. In such instances, one or more of steps 620, 640, 650, and 635 may be deleted.

FIG. 7 is a flow-chart of a second embodiment of a method of programming a stimulation device 300. As described in FIG. 6, the method of FIG. 7 begins after implantation of a lead. Tissue is stimulated (step 710) at a first position of the centroid of stimulation, S. In some embodiments, the stimulation device automatically measures and records data (step 720) at each position. The recorded data may be any input such as impedance measurements, action potential generation, recorded brain signals/waveforms or any combination thereof. The centroid may then be automatically repositioned at the next desired location (step 730). It will be understood that steps 710, 720 and 730 may be repeated until all the regions are stimulated or tested, or until only a predefined region is stimulated or tested. As in the method described in FIG. 6, the data may be displayed (step 740) in any suitable manner. The data may also be stored (step 750) in a memory for future analysis.

In some embodiments, the programming unit 370 includes software that accepts inputs from both the user and recorded data. For example, after stimulation at a selected position, the stimulation device 300 may automatically record an impedance measurement as well as prompt a user for an input, such as for example, the presence of any side effects as described with reference to step 620. The programming unit 370 may then display (step 740) or store (step 750) the stimulation profile and the associated user-input data as well as the data automatically recorded by the stimulation device 300.

It will be understood that the lead may also be used to provide stimulation as therapy with the repositioning of the centroid of stimulation. In such instances, one or more of steps 720, 740, and 750 may be deleted.

It will be understood that each block of the flowchart illustrations, and combinations of blocks in the flowchart illustrations, as well any portion of the stimulation device, implantable pulse generator, lead, systems and methods disclosed herein, can be implemented by computer program instructions. These program instructions may be provided to a processor to produce a machine, such that the instructions, which execute on the processor, create means for implementing the actions specified in the flowchart block or blocks or described for stimulation device, implantable pulse generator, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

Modifications of these methods are possible. For example, by varying the size and shape of the segmented electrodes 130, it may be possible to produce leads capable of applying different stimulation and recording advantages. Moreover, in some embodiments, the centroid of stimulation, S, travels in a path that is defined by any suitable curve or path. In some embodiments, these methods are used with lead constructions other than deep brain stimulation leads.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A method of treating a target region in the brain, the method comprising:
a) contacting tissue to be stimulated with a lead of a stimulation device, the stimulation device comprising a pulse generator coupled to the lead, the lead having a plurality of segmented electrodes disposed at a distal end of the lead, the stimulation device being configured and arranged to stimulate a target region using a positionable centroid of stimulation;
b) providing stimulation current to at least one of the segmented electrodes of the lead to generate a centroid of stimulation at a location and stimulate tissue around the location of the centroid of stimulation;
c) repositioning the centroid of stimulation to a next location along a helical path by altering the provision of stimulation current to the plurality of electrodes and stimulating tissue around the location of the repositioned centroid of stimulation; and
d) repeating c) for each location along the helical path.

2. The method of claim 1, further comprising
collecting data associated with each of the locations of the centroid of stimulation; and
displaying at least a portion of the collected data.

3. The method of claim 2, further comprising storing the collected data in a memory.

4. The method of claim 2, wherein collecting data comprises manually entering data relating to the stimulation.

5. The method of claim 4, wherein the data comprises data relating to side effects.

6. The method of claim 4, wherein the data comprises data relating to the success of stimulation.

7. The method of claim 4, wherein the data comprises data relating to a level of parasthesia.

8. The method of claim 2, wherein collecting data associated with the location comprises automatically collecting and recording data relating to stimulation.

9. The method of claim 8, wherein the data comprises an impedance measurement.

10. The method of claim 8, wherein the data comprises data relating to action potential generation.

11. The method of claim 8, wherein the data comprises data relating to brain activity.

12. The method of claim 2, wherein displaying at least a portion of the collected data comprises producing one or more of a graph, a chart, a plot or a spreadsheet.

13. The method of claim 1, wherein repositioning the centroid of stimulation comprises using a telemetry unit to alter the provision of stimulation current to the plurality of electrodes.

14. The method of claim 13, wherein repositioning the centroid of stimulation comprises repositioning the centroid of stimulation to a next location along the helical path that is closer to the distal end of the lead.

15. The method of claim 13, wherein repositioning the centroid of stimulation comprises repositioning the centroid of stimulation to a next location along a helical path that is closer to the proximal end of the lead.

16. The method of claim 1, further comprising adjusting a parameter of stimulation after stimulating tissue at the location.

17. A computer-readable medium having processor-executable instructions for stimulating tissue, the processor-executable instructions when installed onto a stimulation device enable the stimulation device to perform actions, the stimulation device comprising a pulse generator coupleable to a lead having a plurality of segmented electrodes disposed at a distal end of the lead, the stimulation device being configured and arranged to stimulate a target region using a positionable centroid of stimulation, the actions comprising:
a) providing stimulation current to at least one of the segmented electrodes of the lead to generate a centroid of stimulation at a location and stimulate tissue around the location of the centroid of stimulation;
b) repositioning the centroid of stimulation to a next location along a helical path by altering the provision of stimulation current to the plurality of electrodes and stimulating tissue around the location of the repositioned centroid of stimulation; and
c) repeating b) for each location along the helical path.

18. The computer-readable medium of claim 17, wherein the actions further comprise
collecting data associated with each of the locations of the centroid of stimulation; and
displaying at least a portion of the collected data.

19. The computer-readable medium of claim 17, wherein the actions further comprise adjusting a parameter of stimulation after stimulating tissue at the location.

20. The computer-readable medium of claim 18, wherein the actions further comprise storing the collected data in a memory.

21. A stimulation device, comprising:
a pulse generator coupleable to a lead having a plurality of segmented electrodes disposed at a distal end of the lead, the stimulation device being configured and arranged to stimulate a target region using a positionable centroid of stimulation, and a processor for executing processor-readable instructions that enable actions, including:
a) providing stimulation current to at least one of the segmented electrodes of the lead to generate a centroid of stimulation at a location and stimulate tissue around the location of the centroid of stimulation;
b) repositioning the centroid of stimulation to a next location along a helical path by altering the provision of stimulation current to the plurality of electrodes and stimulating tissue around the location of the repositioned centroid of stimulation; and
c) repeating d) for each location along the helical path.
